(19) United States
(12) Patent Application Publication
(10) Pub. No.: US 2003/0049239 A1
(43) Pub. Date: Mar. 13, 2003

(54) Therapeutic compositions and methods for treating tumors

(76) Inventors: Mariam Grigorian, Copenhagen (DK); Eugene M. Lukanidin, Copenhagen (DK)

Correspondence Address: SCULLY SCOTT MURPHY & PRESSER, PC, 400 GARDEN CITY PLAZA, GARDEN CITY, NY, 11530

(21) Appl. No.: 10/301,328

(22) Filed: Nov. 21, 2002

Related U.S. Application Data
(63) Continuation of application No. 09/553,533, filed on Apr. 20, 2000.
(60) Provisional application No. 60/130,889, filed on Apr. 23, 1999.

(51) Int. Cl. A61K 48/00; A61K 38/48; C12N 9/02

(52) U.S. Cl. 424/93.21; 424/94.63; 435/189; 514/44

(57) ABSTRACT

According to the present invention, Mts-1 interferes with the function of tumor suppressor p53 by binding to p53. The present inventors have demonstrated that binding of Mts-1 inhibits p53 phosphorylation by PKC, represses the DNA-binding activity of p53 and reduces the transactivation capacity of p53. The present invention has further identified that the Mts-1 protein binds to the C-terminal region of p53. Accordingly, the present invention provides compositions and methods useful for treating tumors by, e.g., intercepting the interaction between Mts-1 and p53, or inhibiting the expression or function of Mts-1.

### CROSS REFERENCE TO RELATED APPLICATIONS

This application claims priority from U.S. Provisional Application No. 60/130,889, filed on Apr. 23, 1999.

### FIELD OF THE INVENTION

This invention relates to cancer therapeutics. More particularly, this invention relates to the interception of the Mts-1 binding to p53, which binding prevents p53 from functioning as a tumor suppressor.

### BACKGROUND OF THE INVENTION

p53 is a tumor suppressor protein found in humans and other mammals (See, e.g., Harris, Science 262: 1980-1981, 1993). The wild-type p53 protein functions to regulate cell proliferation and cell death (also known as apoptosis). While the mechanism through which the wild-type p53 protein suppresses tumor cell growth is not completely defined, it is known that one key feature of the growth suppression is the capacity of p53 to act as a transcription factor (Farmer et al., Nature 358, 83-86, 1992; and Kern et al., Science 256, 827-830, 1992).

The nucleotide and amino acid sequences of human p53 have been reported by Zakut-Houri et al, EMBO J. 4: 1251-1255, 1985). The ability of p53 to bind DNA in a sequence-specific manner maps to amino acid residues 90-290 of human p53 (Pavletich et al, Genes Dev. 7: 2556-2564, 1993; and Wang et al, Genes Dev. 7: 2575-2586 1993); the tetramerization domain maps to amino acid residues 32.2-355 of human p53. The DNA binding-regulation domain maps to amino acid residues 364-393 of human p53 or to the corresponding region encompassing residues 361-390 of mouse p53 (Hupp et al., Cell 71: 875-886, 1992; and Halazonetis et al., EMBO J. 12: 1021-1028, 1993).

Inactivation of p53 is associated with more than half of all human tumors. The inactivation can occur by mutation of the p53 gene or through binding of p53 to viral or cellular oncogene proteins, such as the SV40 large T antigen and MDM2. Mutations of the p53 protein in most human tumors involve the sequence-specific DNA binding domain (Bargonetti et al., Genes Dev. 6: 1886-1898, 1992).

Introduction of wild-type or modified p53 into tumor cells has been proposed to as an approach to treat human cancer. See, e.g., T. Fujiwara et al., Current Opinion in Oncology 6: 96-105 (1994); T. Friedmann, Cancer 70: 1810-1817 (1992); U.S. Pat. Nos. 5,847,083 and 5,747,469.

Mouse, rat and human Mts-1 genes have been previously identified and isolated (Linzer et al., Proc. Natl. Acad.Sci. USA 80: 4271-4275, 1983, Barraclogh et al., J. Mol. Biol. 198: 13-20, 1987 and U.S. Pat. No. 5, 798,257 to Zain et al.). The Mts-1 protein, as a calcium binding protein, is believed to have a role in cell growth and myoepithelial cell differentiation. U.S. Pat. No. 5,798,257 further discloses that the mammalian Mts-1 gene is expressed at 10-100 fold higher levels in metastatic cells compared to non-metastatic cells and normal cells, and thus, that the increased expression of Mts-1 within a cell or tissue is indicative of metastatic cancer.

The present invention has identified the tumor suppressor protein p53 as a target for the metastasis associated Mts1 protein.

### SUMMARY OF THE INVENTION

One aspect of the invention is directed to methods of identifying a compound which interferes with the interaction between Mts-1 and p53 by binding to Mts-1 (i.e., binding-intercepting compounds).

Compounds so identified by the screening methods of the present invention form another aspect of the present invention.

Another embodiment of the present invention provides a method for intercepting the binding between p53 and Mts-1 in a subject by administering to the subject, an effective amount of a peptide which prevents the interaction between p53 and Mts-1 by binding to Mts-1. For example, one such peptide comprises the C-terminal region of p53 (amino acid 289-393 of human p53 or amino acid 289-390 of murine p53), in particular, amino acid 360-393 of human p53 or amino acid 360-390 of murine p53. Functional fragments or analogs of such peptides are also within the scope of the present invention. Another example of a binding intercepting peptide comprises amino acid 1909-1937 of non-muscle myosin heavy chain or functional fragments of analogs thereof.

Another embodiment of the present invention provides a method for intercepting the binding between p53 and Mts-1 in a subject by administering to the subject, an effective amount of a nucleic acid molecule coding for a peptide which prevents the binding between p53 and Mts-1.

Another embodiment of the present invention provides a method for intercepting the binding between p53 and Mts-1 in a subject by administering to the subject, an effective amount of an anti-Mts1 antibody which prevents the binding between p53 and Mts-1.

In one embodiment, the present invention provides methods of treating a tumor in a subject by administering to the subject, a therapeutically effective amount of a nucleic acid molecule coding for a peptide which prevents the binding of Mts-1 to p53.

In one embodiment, the present invention provides methods of treating a tumor in a subject by administering to the subject, a therapeutically effective amount of a peptide which prevents the binding of Mts-1 to p53.

In another embodiment, the present invention provides a method of treating a tumor in a subject by administering to the subject, a therapeutically effective amount of an antibody directed against Mts-1.

In still another embodiment, the present invention provides methods of treating a tumor in a subject by administering a therapeutically effective amount of an antisense DNA of Mts-1 gene.

### DESCRIPTION OF DRAWINGS

FIG. 1 depicts co-immunoprecipitation of Mts1 and p53.

Top panel—CSML-100 lysates immunoprecipitated by different batches of anti-Mts1 antibody (1-4) and control antibody (C). Antibodies 3 and 4 effectively pulled down Mts1-protein complexes with Myosin (200 kDa) and p53 (53 kDa). Antibodies 1 and 4 are less effective for complex IP.

Bottom panel—Cells were metabolically labelled with ³⁵S-Methionin and immunoprecipitation was performed using: lysate from CSML-100 cells+anti-Mts1 serum (lane 1), lysate from CSML-100 cells+control antibody (lane 2), lysate from CSML-0 cells+pAb 421 (lane 3), lysate from CSML-100 cells+pAb 421 (lane 4).

FIG. 2 depicts immunoprecipitation of non-labeled Mts-1 from CSML-100 cells using anti-p53 antibodies directed to various epitopes.

FIG. 3 depicts the interaction between Mts1 and the C-terminal domain of p53. Full size recombinant p53 and its domains were mixed with recombinant Mts1 and pooled-down with anti-p53 antibodies. Western blot was performed with immunoprecipitates followed by immunoprobing with anti-Mts1 antibody. Lanes 1,2-full size p53; 3,4-N-terminal domain; 5,6-DNA-binding domain; 7,8-C-terminal domain; and in 1,3,5 and 7-control antibody was used.

FIG. 4 depicts binding of the recombinant Mts1 to p53-GST fusion proteins fixed on Glutahione-sepharose beads. Mts1 associated with GST (negative control), GST-p53 (wild type p53) and GST-p53-Δ30 (mutated p53 lacking amino acids 364-393) was analyzed by Western blot with following immunoprobing with anti-mts1 antibody.

FIG. 5 depicts Mts1 interaction with target proteins in a blot-overlay assay. Recombinant full size p53 (1), N-terminal domain (2), DNA-binding domain (3), C-terminal domain (4) and the fragment of the non-muscle myosin (5) after gel electrophoresis were transferred onto nitrocellulose membrane. Identical membranes were incubated with different batches of the recombinant Mts1 protein (Mts1-a, Mts1-b, Mts1-c and Mts1-d). Mts1 bound to the fixed proteins was detected by the anti-Mts1 serum. The graph at the upper left depicts the schematic localization of the proteins on the membranes.

FIG. 6 depicts the Mts-1 inhibition of p53 phosphorylation by PKC on the C-terminal domain in vitro.

Top—phosphorylation of full size recombinant p53 in the presence and absence of recombinant Mts1.

Bottom—phosphorylation of the N-terminal domain (left), the DNA-binding domain (middle) and the C-terminal domain of P53 in presence of Mts1 (with increasing Mts concentrations from left lanes to right lanes in each radiography).

FIG. 7 depicts phosphorylation by CKII full size p53 (lanes 1,2) and C-terminal domain of p53 (lanes 3,4) in the absence (lanes 1,3) and presence (lanes 2,4) of the recombinant Mts1. Left panel: autoradiography of the dried gel; right panel—Coomassie staining of the same gel.

FIG. 8 depicts EMSA using CSML-0 nuclear extracts with p53 binding site from p21/WAF1 promoter.

FIG. 9A depicts the inhibition by Mts1 of the p53 transactivation of the p21/WAF2-luciferase reporter gene in CSML-0 cells. CSML-0 cells were transiently transfected with p21-luc along with p53 and mts1. Cells were collected in 24 hours and relative luciferase activity was determined.

FIG. 9B depicts transactivation of p53-responsive reporters in Sao-2 cells. Cells were transiently transfected with p21-luc along with p53 and/or mts1. Cells were collected in 24 hours and relative luciferase activity was determined.

FIG. 10 depicts the effects of anti-Mts1 antibody administered to mice bearing highly metastatic CSML-100 tumors.

### DETAILED DESCRIPTION OF THE INVENTION

In accordance with the present invention, Mts-1 interferes with the function of tumor suppressor p53 by binding to p53. The present inventors have demonstrated that binding with Mts-1 inhibits p53 phosphorylation by PKC, represses the DNA-binding activity of p53 and reduces the transactivation capacity of p53. The present invention has further identified that the Mts-1 protein binds to the C-terminal region of p53. Accordingly, the present invention provides compositions and methods useful for treating tumors by, e.g., intercepting the interaction between Mts-1 and p53, or inhibiting the expression or function of Mts-1.

One aspect of the invention is directed to methods of identifying a compound which prevents the interaction between Mts-1 and p53 by binding to Mts-1, also referred to as “a binding intercepting compound”.

The term “compound” is taken to include both organic compounds such as peptides, as well as inorganic compounds such as ion chelators. Antibodies, e.g., polyclonal or monoclonal antibodies directed against Mts-1, the Fab, Fab′, F(ab′)₂ fragments of such antibodies, as well as single-chain anti-Mts-1 antibodies can also be considered as compounds of the present methods.

Preferred test compounds to be screened by the present methods are peptides which are made to resemble the Mts-1 binding site on p53.

“The Mts-1 binding site” refers to the part on a p53 protein which interacts with a Mts-1 protein and is responsible for the binding of the Mts-1 protein to the p53 protein. The binding site is constituted by at least about 6, preferably at least about 8 or 9, or more preferably, at least about 12, amino acid residues of p53 which are contiguous in the primary sequence. Alternatively, the amino acids constituting the binding site are non-contiguous in the primary sequence, but are in the functional vicinity of each other in the tertiary structure of p53. Those skilled in the art can map the Mts-1 binding site on p53 by using a variety of modern molecular biology techniques, such as systematic mutagenesis in combination with 3-D modeling.

In a preferred embodiment, the test peptides to be screened in the present methods are fragments of p53 or analogs thereof, in particular, fragments of the C-terminal domain of p53 or analogs thereof, e.g., fragments of amino acid 289-390 or 360-390 of mouse p53 or fragments of amino acid 289-393 or 360-393 of human p53.

Other preferred peptides to be screened in the present methods are fragments of nonmuscle myosin heavy chain or analogs thereof, in particular, fragments of the C-terminal nonmuscle myosin heavy chain or analogs thereof, e.g., fragments of amino acid 1909-1937 of human nonmuscle myosin heavy chain.

According to the present invention, a peptide fragment can be as short as 6 amino acids in length, preferably, at least about 8 amino acid in length, more preferably, at least about 12 amino acid in length, to mimic the binding site on p53.

By “analogs” it means variants of a peptide in issue. The variations include substitutions, insertions or deletions of one or more amino acid residues, or modifications of the side chains of the amino acid residues. Thus, analogs of a peptide can include homologous peptides from other mammalian species, peptides containing non-natural amino acid residues, peptides having chemical modifications on the side groups of amino acid residues, as well as peptides artificially designed to resemble the three dimensional structure of the binding site on human p53.

A variety of techniques are available to those skilled in the art to make various fragments or analogs of p53. Such techniques include standard chemical synthesis, recombinant expression, and structural modeling (also called ‘mimetics’). The sequences of p53 from a number of mammalian species are highly conserved and are available to those skilled in the art, e.g., via Databases such as GenBank.

Mimetics is a well-known technique involving the design of compounds which contain functional groups arranged in a manner mimicking that of the original, lead compound. Mimetics is desirable where the synthesis of the original compound is difficult, or where the original compound is unsuitable for a particular method of administration (e.g., orally) as such compound may tend to be degraded too quickly by proteases in the alimentary canal. Mimetic design, synthesis and testing can avoid laborious screenings of a large number of molecules for a target property.

To identify a compound which prevents the binding of Mts-1 to p53, a sample containing Mts-1 proteins can be contacted with a test compound for a period of time sufficient to allow binding of the test compound to Mts-1. Then, the sample is contacted with p53, and the amount of complexes formed between Mts-1 and p53 can be measured and compared to the amount of complexes formed in the absence of the test compound. A decrease in the value determines the test compound as a compound which prevents the binding of Mts-1 to p53.

An alternative format to carry out the method of the present invention can include as a first step, contacting a sample containing Mts-1 with p53 for a time sufficient to allow p53 to bind to Mts-1 and measuring the amount of complexes formed between Mts-1 and p53 in the sample. Afterwards, the sample is contacted with a test compound for an appropriate period of time. Compounds which displace p53 from the prior formed p53-Mts1 complexes can be identified as a compound which prevents the binding of Mts-1 to p53.

A variety of biochemical assays and immunoassays can be employed for measuring the amount of the Mts1-p53 complexes formed in a sample. The proteins can be conveniently labeled with, e.g., isotope, biotinyl group, or fluorescein, to facilitate the detection. The assays can be carried out in a variety of formats, such as immunoprecipitation, Far Western blot analysis, RIA, ELISA, forward or reverse sandwich assays, peptide competition binding assays, and the like.

The assays can be readily adapted to provide screens at a large scale, for example, from synthetic combinatorial peptide libraries, by carrying out the process in a 96-well format. Automated screening techniques can be applied in these circumstances as would be understood in the art.

Compounds identified by using any of the above-described biochemical assays or immunoassays can be further tested and confirmed in functional assays, such as DNA-binding gel shift assay, or a reporter expression assay, which are further described in the Examples hereinafter.

Compounds so identified by the screening methods of the present invention form another aspect of the present invention.

In particular, the present invention provides preferred compounds which prevent p53 from binding to Mts1, e.g., a peptide comprising aa 289-393 of human p53, a peptide comprising aa 360-393 of human p53, a peptide comprising aa 289-390 of murine p53, a peptide comprising aa 360-390 of murine p53, a peptide comprising the C-terminal nonmuscle myosin heavy chain, a peptide comprising amino acid 1909-1937 of human nonmuscle myosin heavy chain. Functional fragments and analogs of these peptides are also contemplated by the present invention.

“Functional fragments or analogs” refer to peptide fragments or analogs that have the same function as the peptide in issue, namely, the function of interfering the Mts1-p53 interaction by binding to Mts-1. Such fragments and analogs can be made and identified as described hereinabove.

The present invention also contemplates pharmaceutical compositions which include, as an active ingredient, an Mts1-p53 binding intercepting compound as described hereinabove.

Another embodiment of the present invention provides a method for intercepting the binding between p53 and Mts-1 in a subject by administering to the subject, an effective amount of a peptide which prevents the binding between p53 and Mts-1. Preferred intercepting peptides and fragments or analogs thereof have been described hereinabove.

Another embodiment of the present invention provides a method for intercepting the binding between p53 and Mts-1 in a subject by administering to the subject, an effective amount of a nucleic acid molecule coding for a peptide which prevents the binding between p53 and Mts-1.

Preferably, such nucleotide sequence is provided in an expression vector. Preferred expression vectors for use in a therapeutic composition include any appropriate gene therapy vectors, such as nonviral (e.g., plasmid vectors), retroviral, adenoviral, herpes simplex viral, adeno-associated viral, polio viruses and vaccinia vectors. Examples of retroviral vectors include, but are not limited to, Moloney murine leukemia virus (MoMuLV), Harvey murine sarcoma virus (HaMuSV), murine mammary tumor virus (MuMTV), and Rous Sarcoma Virus (RSV)-derived recombinant vectors. More preferably, a non-human primate retroviral vector is employed, such as the gibbon ape leukemia virus (GaLV), thereby providing a broader host range than murine vectors, for example. Gene therapy vectors can be made tissue specific by, for example, linking the nucleotide sequence to a tissue-specific promoter. Multiple teachings of gene therapy are available for those skilled in the art, e.g., W. F. Anderson (1984) “Prospects for Human Gene Therapy” Science 226: 401-409; S. H. Hughes (1988) “Introduction” Current Communications in Molecular Biology 71: 1-12; N. Muzyczka and S. McLaughlin (1988) “Use of Adeno-associated Virus as a Mammalian Transduction Vector” Communications in Molecular Biology 70: 39-44; T. Friedman (1989) “Progress Toward Human Gene Therapy” Science 244: 1275-1281 and W. F. Anderson (1992) “Human Gene Therapy” Science 256: 608-613.

The nucleic acid molecule can be delivered “naked” by direct injection into the blood stream or to the desired tissue or organ of a subject. Alternatively, the vector can be combined with a lipid compound which facilitates the uptake of the molecule by cells. The lipid compound include liposome, lipofectins, cytofectins, lipid-based positive ions, and then introduced into the body fluids, the blood stream, or a selected tissue site. Liposome mediated gene therapy is well known in the art and is described by, e.g., Lesoon-Wood et al., Human Gene Ther. 6: 395, 1995; Tsan et al., Am. J. Physiol 268: 11052, 1995; Vieweg et al., Cancer Res. 5585: 2366, 1995; Trivedi et al., J. Neurochem. 64: 2230, 1995; Hickman et al., Human Gene Ther. 5: 1477, 1994; Westbrook et al. Human Mol Genet. 3: 2005, 1994; Schmid et al., Z. Gastroenterol 32: 665, 1994; Hofland et al., Biochem. Biophys. Res. Commun. 207: 492, 1995; Plautz et al., Ann. N.Y. Acad. Sci. 7168: 144, 1994. Other DNA carriers which can facilitate the uptake of a desired vector by the target cells include nuclear protein, or ligands for certain cell receptors, which can be combined with a vector in engineered vesicles for delivery.

Another embodiment of the present invention provides a method for intercepting the binding between p53 and Mts-1 in a subject by administering to the subject, an effective amount of an anti-Mts1 antibody which prevents the binding between p53 and Mts-1. Polyclonal or monoclonal antibodies directed against Mts-1, the Fab, Fab′, F(ab′)₂ fragments of such antibodies, as well as single-chain anti-Mts-1 antibodies can all be employed.

In another aspect of the present invention, Mts1-p53 binding-intercepting peptides or nucleic acid molecules encoding thereof are used for treating tumors.

By “treating a tumor” it means that the tumor growth or metastasis is significantly inhibited, as indicated by reduced tumor volumn or reduced occurrences of tumor metastasis. Tumor growth can be determined, e.g., by examining the tumor volume via routine procedures (such as obtaining two-dimensional measurements with a dial caliper). Tumor metastasis can be determined by examining the appearance of tumor cells in secondary sites or examining the metastatic potential of biopsied tumor cells in vitro using various laboratory procedures.

According to the present invention, the tumors which can be treated by using the methods of the present invention may include, but are not limited to, melanoma, lymphoma, plasmocytoma, sarcoma, glioma, thymoma, leukemia, breast cancer, prostate cancer, colon cancer, esophageal cancer, brain cancer, lung cancer, ovary cancer, cervical cancer, hepatoma, and other neoplasms known in the art.

The present invention contemplates particularly p53-related tumors. The term “p53-related” refers to tumor cells in which wild-type (wt) p53 is absent, disabled or otherwise mutated.

A variety of methods are available to those skilled in the art for determining whether a tumor is “p53-related”. For example, EPA 518 650 (Vogelstein, B. et al.) describes a method for detecting p53 in cellular extracts using DNA sequences that are specific for p53 binding. WO 94/11533 describes determining the presence of functional p53 in cells by measuring mRNA or protein encoded by a growth-arrest and DNA-damage inducible gene, GADD45. U.S. Pat. No. 5,876,711 describes a rapid in vivo method for determining the status of tumor suppressor proteins in patient tumor cells. Such method includes contacting the tumor cells with a first and second polynucleotide sequence such that they are taken up by the tumor cells. The first polynucleotide sequence encodes a reporter molecule that is operably linked to the second polynucleotide sequence which sequence binds the tumor suppressor. Binding of the tumor suppressor causes the expression of the reporter molecule, which is then detected or quantitated.

In one embodiment, the present invention provides methods of treating a tumor in a subject by administering to the subject, a therapeutically effective amount of a peptide which prevents the binding of Mts-1 to p53. Preferred binding intercepting peptides have been described hereinabove.

In another embodiment, the present invention provides methods of treating a tumor in a subject by administering to the subject, a therapeutically effective amount of a nucleic acid molecule coding for a peptide which prevents the binding of Mts-1 to p53.

In a further aspect of the invention, methods of treating a tumor are provided which are based on inactivating, sequesting, or interfering the function of Mts proteins or abolishing the expression of the Mts-1 gene.

In one embodiment, the present invention provides a method of treating a tumor in a subject by administering to the subject, a therapeutically effective amount of an antibody directed against Mts-1.

The present invention provides, as an example, that the administration of an anti-Mts-1 monoclonal antibody to mice bearing highly metastatic CSML-100 tumors has a significant inhibitory effect on both tumor growth and metastasis.

Both monoclonal and polyclonal antibodies directed against a mammal Mts-1 protein, including rat, mouse and human, can be employed for practicing the methods of the present invention. Such antibodies can be readily generated using the entire Mts-1 protein as an antigen or by using short peptides, encoding portions of the Mts-1 protein, as antigens. Preferably, specific peptides encoding unique portions of the Mts gene are synthesized for use as antigens for obtaining anti-Mts1 antibodies. Those skilled in the art can refer to U.S. Pat. No. 5,801,142 for relevant teachings.

In still another embodiment, the present invention provides methods of treating a tumor in a subject by administering a therapeutically effective amount of an antisense DNA of Mts-1 gene.

A Mts-1 antisnese DNA can have at least about 10 nucleotides, preferably, at least about 15 or 17 nucleotides, more preferably, at least about 50 nucleotides. The antisense DNA is preferably inserted into an expression vector in an operable linkage to a promoter which can effect the transcription of the antisense RNA. Any of the foregoing gene therapy vectors can be used for practicing the methods of the present invention.

In practicing the above-described methods of the present invention, the active compound (i.e., the binding-intercepting peptides, the nucleic acid molecules encoding such peptides, anti-Mts1 antibodies, or Mts-1 antisense DNAs) can be used in combination with one another, or with other anti-tumor agents that are available in the art.

The active compounds can be suitably administered in combination with pharmaceutically acceptable carriers. The carrier can be liquid, semi-solid, e.g. pastes, or solid carriers. Except insofar as any conventional media, agent, diluent or carrier is detrimental to the recipient or to the therapeutic effectiveness of the active ingredient contained therein, its use in practicing the methods of the present invention is appropriate. Examples of carriers include fats, oils, water, saline solutions, lipids, liposomes, resins, binders, fillers and the like, or combinations thereof.

In accordance with the present invention, the active ingredients can be combined with the carrier in any convenient and practical manner, e.g., by solution, suspension, emulsification, admixture, encapsulation, absorption and the like, and if necessary, by shaping the combined compositions into pellets or tablets. Such procedures are routine for those skilled in the art.

Dosages of a compound in accordance with the present invention depend on the disease state or condition being treated and other clinical factors, such as weight and condition of the subject, the subject's response to the therapy, the type of formulations and the route of administration. The precise dosage of a compound to be therapeutically effective can be determined by those skilled in the art. As a general rule, the therapeutically effective dosage of a compound can be in the range of about 0.5 μg to about 2 grams per unit dosage form. A unit dosage form refers to physically discrete units suited as unitary dosages for mammalian treatment: each unit containing a pre determined quantity of the active material calculated to produce the desired theraputic effect in association with any required pharmaceutical carrier. The methods of the present invention contemplate single as well as multiple administrations, given either simultaneously or over an extended period of time.

These compounds can be administered via standard routes, including the oral, ophthalmic nasal, topical, parenteral injections (e.g., intravenous, intraperitoneal, intradermal, subcutaneous or intramuscular), as well as direct injection to a preselected tissue site.

All the publications mentioned in the present disclosure are incorporated herein by reference. The terms and expressions which have been employed in the present disclosure are used as terms of description and not of limitation, and there is no intention in the use of such terms and expressions of excluding any equivalents of the features shown and described or portions thereof, it being recognized that various modifications are possible within the scope of the invention.

### EXAMPLE 1

### Materials and Methods

Plasmids

The coding region of mts1 was cloned in pSK3 (Pharmacia) vector, containing simian virus promoter. The resulting construct was named as pSV-mts1. In pHMG-mts1 the coding part of mts1 with the first intron was cloned in pHMG vector, containing the constitutive promoter of hydroxymethyl-glutaryl-CoA-reductase (HMGCR). PSVBc12 was constructed by cloning the Bc12/Xho1 insert from PEBS7-425 in pSK3 vector.

The following mouse wild type p53 PCRs were designed: #1—full size coding region (390 aa) was amplified using primers: forward CGGGATCCGACTGGATGACTGCCATGGA (having a BamHI site) reverse CGAAGCTTCAGTCTGAGTCAGGCCCCACT (including a HindIII site); #2—N-terminal domain (106 aa): forward, same as the forward primer for #1, and reverse CGAAGTCTTGAAGCCATAGTTGCCCTGGTAAG (including a HindIII site); #3—DNA-binding domain (185 aa): forward CGGGATCCCACCTGGGCTTCCTGCATGCT (including a BamHI site), reverse CGAAGCTTGGACTTCCTTTTTTGCGGAAATTTTC (including a HindIII site); #4—C-terminal (99 aa): forward CGGGATCCCTTTGCCCTGAACTGCCCCCA (including a BamHI site), and reverse—same as the reverse primer for #1. The PCR products were digested with BamHI/HindIII and cloned in eukaryotic expression vector pXmyctag, containing a CMV promoter and 8-aa myc tag, and bacterial expression vector pQE30 (Qiagen). PSP65m65 plasmid DNA was used for the amplification of p53. Human pC53-SN3 (human wild type p53) and pC53-SCX3 (human mutant Human mutant p53-pC53-SCX3 (143^{Val-Ala}) eukaryotic expression plasmids were obtained. For conditional expression, mts1 CDNA was excised, cloned in pUHD 10-3 and used for transfection of cell lines producing reverse tetracycline-controlled transactivator (pUHD172-neo) (Clontech).

p21/WAF-luc was constructed by cloning 13 copies of p53 binding consensus element from the p21/WAF promoter in the pfLUC reporter construct containing the Photinus pyralis luciferase gene under the minimal c-fos promoter Saksela et al. (Mol. Cell. Biol. 13:3698-3705, 1993). The β-galactosidase expression plasmid was purchased from Clontech. pBabe-Hyg contains Hygromicin-resistance gene. pSV2-neo contains neomycin resistance gene.

Cell Lines and Transfection

Mouse mammary adenocarcinoma cell lines: CSML-0 and CSML-100 Senin et al. (Exp. Oncology USSR 5:35-39, 1983), VMR-liv Senin et al. (Vestnik USSR Acad. Med. Sci. 5:85-91, 1984) were derived from two independent spontaneous tumors in A/Sn mice. Saos-2 is a human bsteosarcoma cell line.

Cells were transfected by electroporation: 1-3×10⁶ cells in 100 μl of phosphate saline buffer were transferred into electroporation cuvette and single pulse of 250V and 250 μFd was applied using Bio-Rad electroporation system. Clones were selected in the presence of 400 μg of G-418, for the conventional tetracyline inducible clones, double selection with G-418 and 200 μg/ml Hygromycin was used. In transient transfection experiments, the efficiency of each transfection was monitored by use of a cotransfection of β-galactosidase expression vector, pCMV-gal. At 24-48 hours posttransfection, cells were lysed and the luciferase activity was measured with a luminometer (Promega Corp.). The same lysates were tested for β-galactosidase activity by using o-nitrophenyl-β-galactopyranoside (Sigma) as a chromogenic substrate.

Preparation of Recombinant Proteins

Histidine-tagged p53 and Mts1 proteins were expressed in XL-blue Escherichia coli by induction with 0.2 mM isopropyl β-D-thiogalactopyranoside for 4 hours at 37° C. Protein isolation in denaturating conditions followed by renaturation were performed according to the manufacturer's protocol (Qiagen).

Western Blotting

Protein isolation and western blotting were performed according to Grigorian et al. (Int. J. Cancer 67:831-841, 1996) (IJC). Immunostaining and protein bands visualization with ECL system SuperSignal® (Pierce) were carried out according to the manufacturer's protocol.

Indirect Immunoprecipitations and in vitro Pull-down Assay.

Cells were metabolically labeled for 4 h in methionine-cystein-free medium supplemented with dialyzed and inactivated 10% FCS with 0.2mCi/ml [³⁵S]-methionine and -cystein (Amersham). The cells were lysed in 150 mM NaCl-50 mM Tris-Hcl pH 7.6-0.5% NP-40 and precleaned on 50% protein A-Sepharose. The precleaned lysates were incubated for 2 hours with anti-p53 antibodies: monoclonal pAb421 and goat polyclonal E-19 (Santa Cruz Biotechnology, Inc.) and anti-Mts1 rabbit serum, followed by 5 washes with the same buffer. The precipitated proteins were separated on gradient 4-20% PAAG and detected by autoradiography.

For in vitro pull down assay, 1 μg recombinant Mts1 was mixed with recombinant full size p53 and its domain peptides in 150 mM NaCl-50 mM Tris-HCl pH 8.0-0.5% NP-40 and precleaned on Protein A-Sepharose on the presence of protease inhibitors at 1 hour in cold room. To the precleaned mixtures, fresh portions of the protein A-sepharose and the corresponding anti-p53 antibodies were added: pAb421 for full-size and C-terminal domain, pAb240 for DNA-binding core domain and E-19 for the N-terminal domain, and incubated for 2 hours in the cold room. Following 5 washes, immunoprecipitates were denaturated by heating at 100° C.-5 min, separated in 15% PAAG and transferred to Immobilon-P (Millipore). To detect the co-immunoprecipitated Mts1 protein, membranes were probed with anti-Mts1 antibody and developed by the ECL System. Recombinant human wild type GST-p53 and GST-p53-Δ30 (deletion mutant lacking amino acid residues 364-393) fusion proteins were used for pull down experiments with the Mts1 recombinant protein. 5 μg of GST and GST-fusion proteins coupled with Glutathione-sepharose beads were incubated with 2 μg of the Mts1 protein in NP-40 buffer (1% NP-40, 50 mM Tris-HCl pH 8.0-150 mM NaCl) for 2 h in the cold room with rotation. Beads with proteins bound were washed 5 times with NP-40-buffer. Proteins were isolated by boiling in the protein loading buffer for 5 min and analyzed using Western blotting.

Phosphorylation Assays

Reactions were performed in a mixture (2 μl) containing 50 mM Tris-HCl pH 7.6, 0.2 M NaCl, 10 mM MgCl₂, 4 mM CaCl₂, 2 mM dithiothreitol, 15 μl ATP (Amersham Pharmacia Biotech), 25 μCi [γ-³²P]-ATP (5000 Ci/nnol, Amersham Pharmacia Biotech), 1 μM recombinant wild type p53 or this protein fragments for 30 min at 30° C. PKC assay was done in the presence of 7.5 μg of phosphatidylserine (Sigma) by 0.025 μg PKC (Roche). CKII was purchased from New England BioLabs Inc., and 50 units were applied per each reaction. Recombinant Mts1 was sued in concentrations of 3,5 and 9 μM reactions were terminated by 15% SDS-PAGE. Gels were fixed in 10% trichloracetic acid, dried and exposed to Kodak x-ray film.

Electromobility Shift Assay (EMSA)

Nuclear extracts were prepared as previously described by Kusti ova et al. (Mol. Cell Biol. 12:7095, 1998). To perform EMSA, nuclear extracts were incubated with end-labeled oligonucleotides that contained binding sites for p53 or Oct-1 proteins. Oligonucleotide sequences were as follows: for Oct-1—TGCGAATGCAAATCACTAGAA (LeBowitz J. H., Genes Dev. 2, 1227-1237, 1998); for p53—GAACATGTCCCAACATGTTG, derived from the promoter of p21/WAF Avantaggiati et al. (Cell 89:1175-1184, 1997). The reactions were carried out in 10 μl of the buffer containing 100 mM KCl, 1 mM MgCl₂, 1 mM DTT, 0.1% NP-40, 0.5 mg/ml BSA, 5% glycerol. To perform gel supershift analysis, anti-p53 antibody (pAb421) were added to the EMSA reaction mixtures. The incubation with antibody was carried out for 1 h at 4° C. after the binding reactions were completed.

Northern Blot Analysis

CSML-0 conventional Mts1-tet-inducible clones were grown at low and dense conditions and induced with 2 μg/ml Doxycylin at 0.24,48 and 72 h. RNA was isolated according to Chomczynski et al. (Anal. Biochem. 162:156-159, 1987). Gel elecrophoresis and Northern blot analyses were performed as it is described in Grigorian et al. (Int. J. Cancer 67:831-841, 1996). The filters were sequentially hybridized with murine p21/WAF, Bax and Cyclin G1 probes. The amounts of mRNA on the filters were calibrated by hybridization with γ-³²P-ATP-labeled poly(U) probe. To quantify the intensities of the bands membranes were scanned using a Molecular Dynamics computing densitometer (Sunnyvale, Calif.) with ImageQuant software, after each hybridization.

### EXAMPLE 2

### Mts-1 Binds to the C-Terminal Domain of P53

To determine whether Mts-1 and p53 proteins directly interact with each other, immunoprecipitation(IP) and Far Western experiments were performed. In these experiments, two cell lines were used: CSML-0 cells which express very low level of wt-p53. and does not express Mts1 at all, and CSML100 cells which express mutant p53 and high level of Mts1.

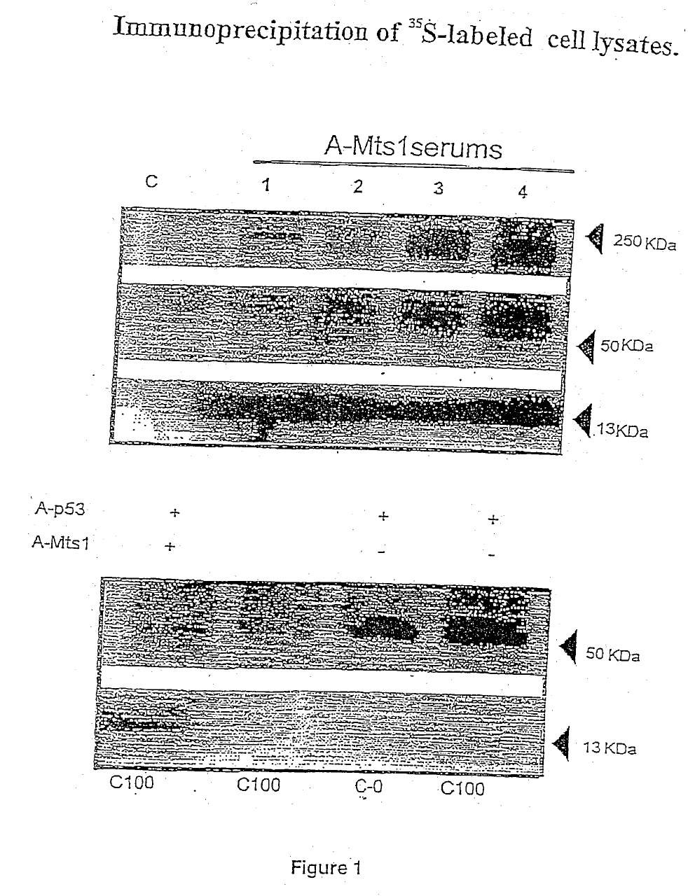

CSML-0 were transfected with tet-inducible Mts1. Lysates from metabolically labeled cells were used for IP with anti-p53 and anti-Mts1 antibodies. As shown in the IP-radioautography assays following SDS-PAGE electrophoresis (FIG. 1), the p53 protein band was readily detected in anti-Mts1 immunoprecipitates and, vice versa, the Mts1 protein band in anti-p53 immunoprecipitates. As a positive control, the band corresponding to the heavy chain of the non-muscle myosin, a known target of Mts1, was also detected in the anti-Mts1 immunoprecipitates.

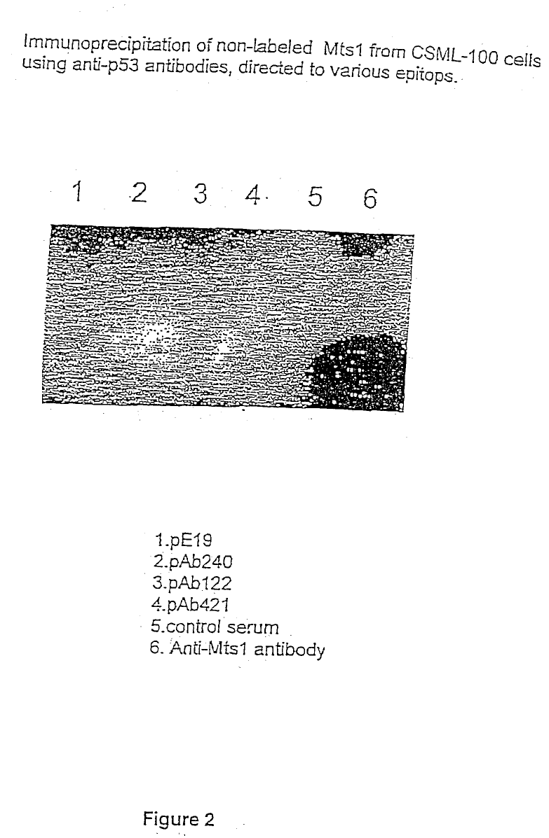

Non-radioactive IP assays with the lysate obtained from 1×.10⁹ CSML-100 cells were performed using several anti-p53 antibodies targeting different epitopes located at N-terminal and C-terminal domains of p53. Immunoprecipitates were subjected to Western blot analysis and probed with anti-Mts1 antibody. The Mts1 protein was easily detected in the complex precipitated by pE19 antibody, directed to the N-terminal domain of p53. The Mts1 protein was not detected in the complexes precipitated by antibodies against C-terminal domain of p53, pAb421 and p122Ab (FIG. 2).

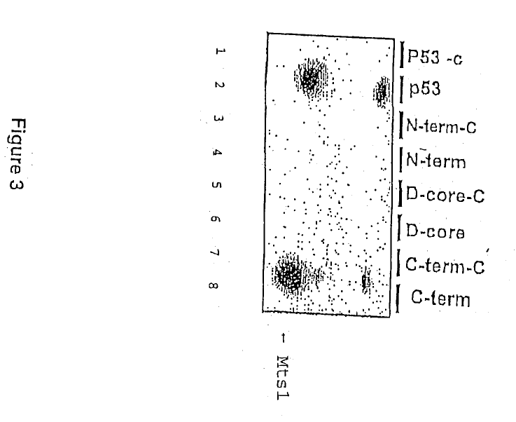

To identify the domain of p53 that interacts with Mts1, the recombinant p53 domains corresponding to N-terminal (transactivation) AA 1-106, core (DNA-binding) AA 104-288 and C-terminal (oligomerization and regulation) AA 289-387 were obtained. Full-size p53 and above mentioned domains were co-immunoprecipated with Mts1 recombinant proteins by anti-p53 antibody. Immunoprecipitates were subjected to Western blot analysis using anti-mts1 antibody. As is shown in FIG. 3, only full-size p53 and the C-terminal domain of p53 immunoprecipitated the Mts1 protein, but not the N-terminal and the DNA-binding domains.

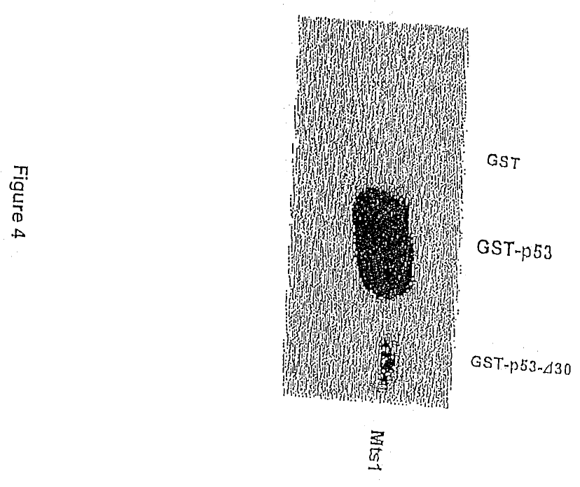

To more precisely map the interaction site, the recombinant wt-p53-GST and Δ30p53-GST (mutant p53 lacking amino acids 364-393 of the C-terminal domain), captured on the Glutathione-Sepharose4B beads, were incubated with recombinant Mts1. Mts proteins bound to wild type or mutant p53 were recovered in SDS-protein loading buffer by boiling followed by PAGE, Western blotting and immunoprobing with anti-Mts1 antibody. FIG. 4 illustrates that p53 molecule with deletion in the C-terminal domain was not able to bind the Mts1 protein, and thus, that the binding site is spread along 364-393 aa of p53.

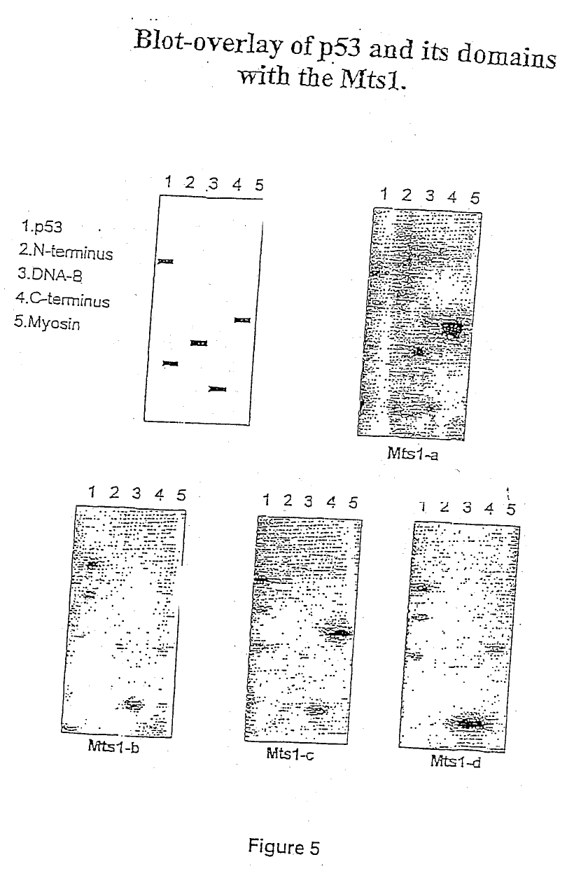

Another approach, Far-Western blot analysis, was also employed to assess the interaction between Mts1 and p53. Full size p53 and its functional domains, expressed in E.coli, were separated on SDS-PAGE and transferred into Immobilon-P. Filters were incubated with recombinant Mts1 in conditions allowing the interaction with the proteins fixed on the membrane. Mts1 bound to p53 proteins on the filter, was detected with anti-Mts1 antibody. Data shown in FIG. 5, consistent with the IP results, indicated that Mts1 was able to bind full-size p53 and its C-terminal domain. As a positive control we have used recombinant fragment of non-muscle myosin which is known as a target for Mts1 protein (FIG. 5, lane 5). BSA loaded in 5× excess did not reveal nonspecific mts1 binding in Far-Western assay, neither did N-terminal or DNA-binding domains.

### EXAMPLE 3

### Mts1 Protein Inhibits Phosphorylation of P53 by PKC

The C-terminal domain of p53 contains PKC and CKII phosphorylation sites. Experiments were carried out to determine whether Mts1 affects the phosphorylation of p53. One micromolar recombinant full-size p53 and its distinct domains were phosphorylated by PKC in the absence and presence of 3, 5 and 9 μM recombinant Mts1 protein, respectively, and subsequently analyzed by SDS-PAGE.

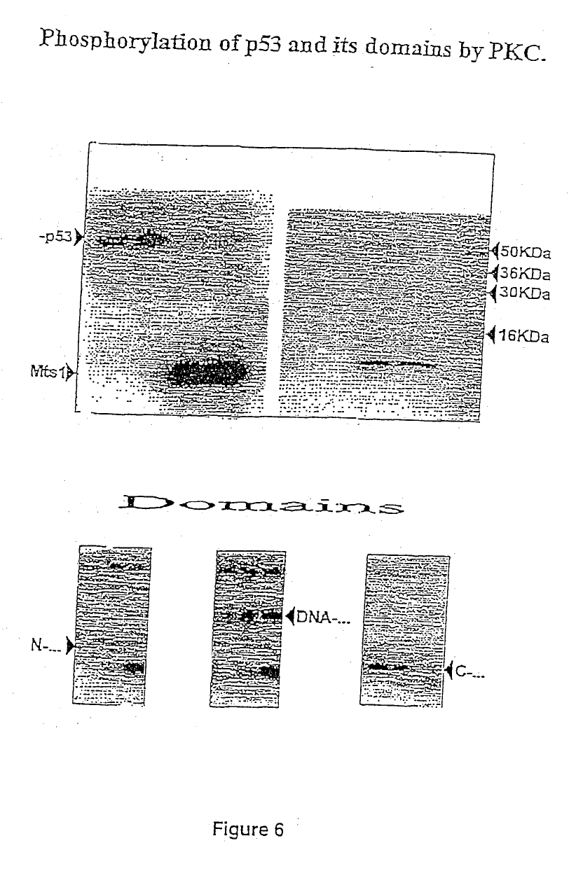

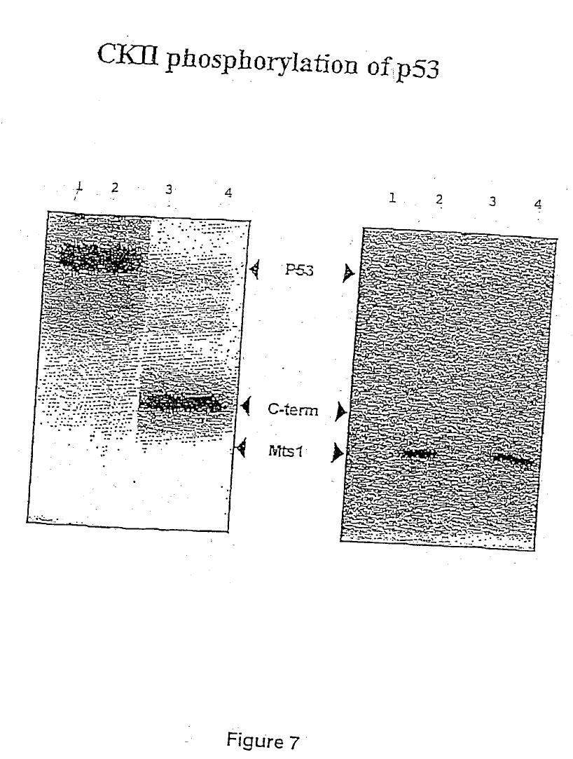

As shown in FIG. 6, Mts-1 inhibited the phosphorylation of full-size p53 and the C-terminal protein fragment by PKC. Addition of the same concentrations of Mts1 to the PKC reaction mixture did not affect the phosphorylation of the N-terminal and DNA-binding domains of p53. No interference of Mts1 was shown with CK II phosphorylation of p53 and its domains (FIG. 7). These observations indicate that Mts1 specifically inhibited the phosphorylation of PKC of the C-terminal domain of p53.

### EXAMPLE 4

### Inhibition of P53 DNA-Binding Activity by Mts1

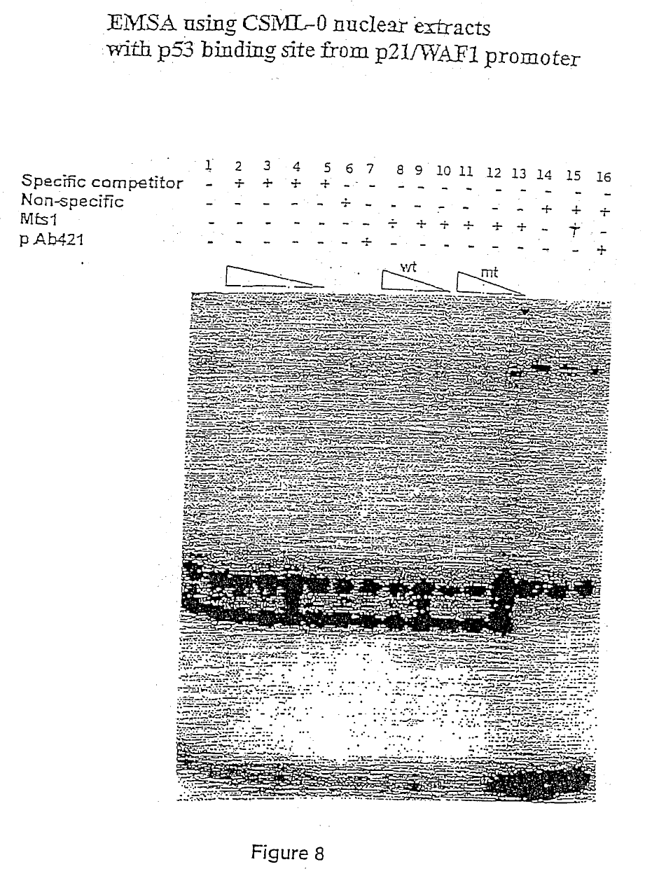

Effects of the Mts1-p53 interaction on the DNA-binding activity of p53 were investigated in an electrophoretic mobility shift assay (EMSA)(FIG. 8).

The end-labeled oligonucleotide containing p53-binding site from p21/WAF promoter was mixed with nuclear extracts containing wild type p53(lanes 1). Specificity of the DNA-protein complexes was confirmed by supershift of the complexes after adding anti-p53 antibody (lanes 7), competition with specific p21/WAF p53 binding site-containing oligonucleotide (lanes 2-5) and absence of influence of non-specific nucleotide (lanes 6,14-16). Incubation of nuclear extracts with the Mts1 protein before adding the labeled oligonucleotide decreased DNA-binding activity of p53 in dose-dependent manner (lanes 8-10). The inhibition was less when Mts1 was added after formation of p53-DNA complexes. Mts1 did not affect the binding Oct-1 factor to oligonucleotide containing Oct-1 binding site (lanes 15).

These results indicate that Mts-1 inhibited the DNA-binding activity of p53.

### EXAMPLE 5

### Mts1 Affects P53-Dependent Transcription

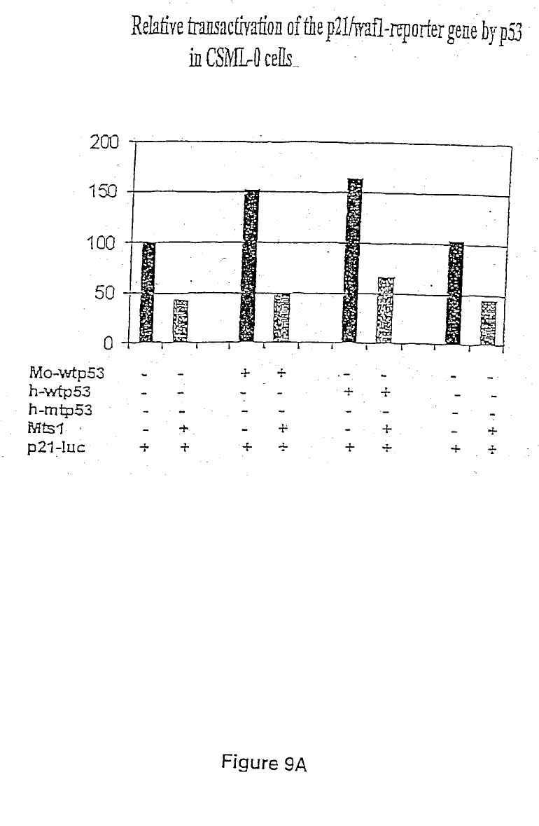

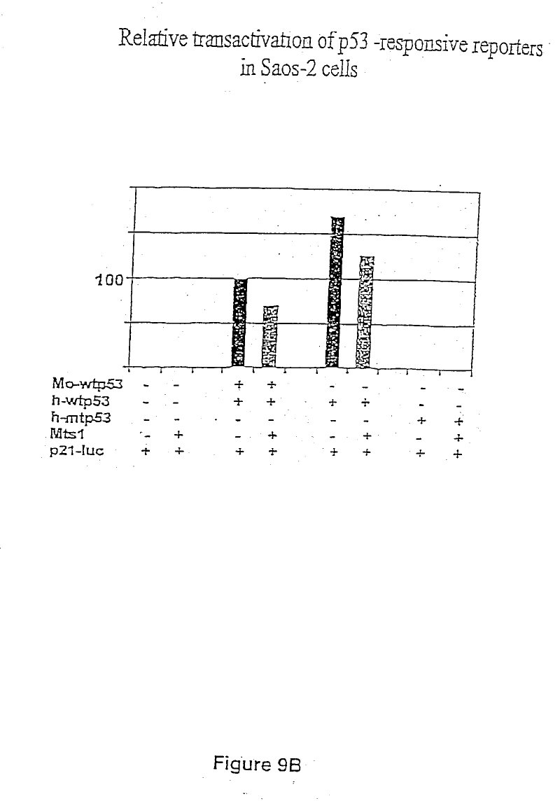

Mts1 CDNA was placed under the control of HMGCG promoter and was cotransfected with constructs bearing p⁵3-binding sites from the p21/WAF promoter, fused with the luciferase reporter gene. Three different mts1-negative cell lines, mouse low (VMR-liv), nonmetastatic (CSML-0) adenocarcinoma cell lines with wtp53 and p53-null Saos-2.1109 cells, were used in this assay. A PCMV-β-gal plasmid was cotransfected for evaluation of the transfection efficiency and the degree of apoptosis. Luciferase activity was adjusted by β-galactosidase activity in each transfection. The results of the 2-3 experiments are summarized in FIGS. 9A and 9B.

The results indicated that Mts1 affected the transactivation activity of p53. In all analyzed cell lines, the presence of Mts1 correlated with the inhibition of the luciferase activity transcribed from the promoter containing the p21/WAF p53-binding consensus. The inhibition was 2-3 folds in CSML-0 and VMR-liv, and 1.3-1.4 fold in Saos-2.1109 cells.

### EXAMPLE 6

### Anti-Mts1 Antibody Enhances Tumor Necrosis and Inhibits Metastasis.

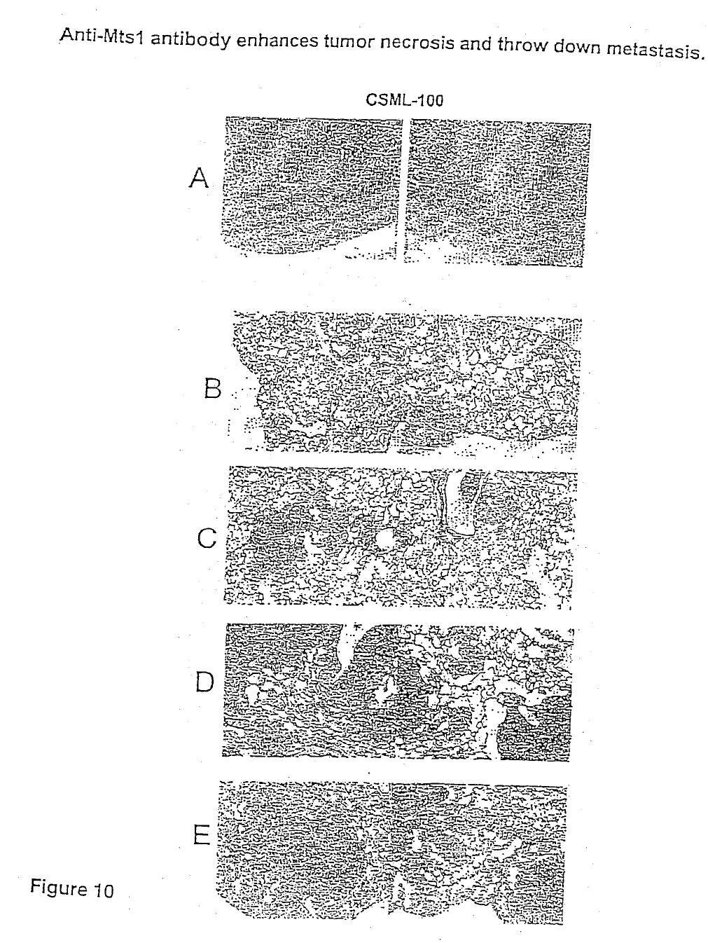

Mice bearing highly metastatic CSML-100 tumors were treated with anti-Mts1 antibody from the initial stages of tumor development via i/v injections In 4 weeks animals were sacrificed. Tumors, lungs and livers were subjected to histological analysis (FIG. 10). Large necrotic areas were observed in tumor mass treated with anti-Mts1 antibody (A,right) compared to tumors treated with control IgG (A,left).

Decrease of metastases was observed in lungs among animals treated with anti-Mts1 antibody (B,C) compared to control animals (D,E).

One experiment with 4 animals in each group was done.

We claim:

1. An isolated peptide, wherein said peptide binds an Mts-1 protein and prevents p53 from binding to Mts-1.

2. The peptide of claim 1, selected from the group consisting of
(1) a peptide comprising amino acid 289-390 of murine p53, or a functional fragment or analog thereof;
(2) a peptide comprising amino acid 289-393 of human p53, or a functional fragment or analog thereof;
(3) a peptide comprising amino acid 360-390 of murine p53 or a functional fragment thereof;
(4) a peptide comprising amino acid 360-393 of human p53 or a functional fragment thereof; and
(5) a peptide comprising amino acid 1909-1937 of human nonmuscle myosin heavy chain.

3. A pharmaceutical composition comprising an isolated peptide and a pharmaceutically acceptable carrier, wherein said peptide binds an Mts-1 protein and prevents p53 from binding to Mts-1.

4. A pharmaceutical composition comprising the peptide of claim 2 and a pharmaceutically acceptable carrier.

5. A pharmaceutical composition comprising a nucleic acid molecule encoding the peptide of claim 2 and a pharmaceutically acceptable carrier.

6. A method of intercepting the binding between p53 and Mts-1 in a subject, which comprises administering to the subject an effective amount of an anti-Mts1 antibody, wherein said antibody prevents the binding between p53 and Mts-1.

7. A method of intercepting the binding between p53 and Mts-1 in a subject, which comprises administering to the subject an effective amount of the peptide of claim 1.

8. A method of intercepting the binding between p53 and Mts-1 in a subject, which comprises administering to the subject an effective amount of the peptide of claim 2.

9. A method of intercepting the binding between p53 and Mts-1 in a subject, which comprises administering to the subject an effective amount of a nucleic acid molecule encoding the peptide of claim 2.

10. The method of claim 9, wherein said nucleic acid molecule is placed in an expression vector selected from a non-viral, retroviral, polio viral, adenoviral, adeno-associated viral, herpes viral, SV 40, or vaccinia vector.

11. A method of treating a tumor in a subject, which comprises administering to the subject, a therapeutically effective amount of the peptide of claim 1 and a pharmaceutically acceptable carrier.

12. A method of treating a tumor in a subject, which comprises administering to the subject an effective amount of the peptide of claim 2 and a pharmaceutically acceptable carrier.

13. A method of A method of treating a tumor in a subject, which comprises administering to the subject an effective amount of a nucleic acid molecule encoding the peptide of claim 2 and a pharmaceutically acceptable carrier.

14. The method of claim 13, wherein said nucleic acid molecule is placed in an expression vector selected from a non-viral, retroviral, polio viral, adenoviral, adeno-associated viral, herpes viral, SV 40, or vaccinia vector.

15. The method of claim 11, 12 or 13, wherein said tumor is one of melanoma, lymphoma, plasmocytoma, sarcoma, glioma, thymoma, leukemia, breast cancer, prostate cancer, colon cancer, esophageal cancer, brain cancer, lung cancer, ovary cancer, cervical cancer, or hepatoma.

16. The method of claim 11, 12 or 13, wherein said tumor is a p53-related tumor.

17. A method of treating a tumor in a subject, which comprises administering to the subject, a therapeutically effective amount of an antibody directed against Mts-1.

18. A method of treating a tumor in a subject, which comprises administering to the subject, a therapeutically effective amount of an antisense DNA of an Mts-1 gene.

19. The method of claim 18, wherein said antisense DNA is placed in an expression vector selected from a non-viral, retroviral, polio viral, adenoviral, adeno-associated viral, herpes viral, SV 40, or vaccinia vector.

20. The method of claim 17 or 18, wherein said tumor is one of melanoma, lymphoma, plasmocytoma, sarcoma, glioma, thymoma, leukemia, breast cancer, prostate cancer, colon cancer, esophageal cancer, brain cancer, lung cancer, ovary cancer, cervical cancer, or hepatoma.

21. A method of identifying a compound which binds an Mts-1 protein and prevents p53 from binding to Mts-1.


SEQUENCE LISTING (8 sequences)

SEQ ID NO: 1 - DNA, 28 bp, Artificial Sequence
  Description of Artificial Sequenceprimer
cgggatccgactggatgactgccatgga

SEQ ID NO: 2 - DNA, 29 bp, Artificial Sequence
  Description of Artificial Sequenceprimer
cgaagcttcagtctgagtcaggccccact

SEQ ID NO: 3 - DNA, 32 bp, Artificial Sequence
  Description of Artificial Sequenceprimer
cgaagtcttgaagccatagttgccctggtaag

SEQ ID NO: 4 - DNA, 29 bp, Artificial Sequence
  Description of Artificial Sequenceprimer
cgggatcccacctgggcttcctgcatgct

SEQ ID NO: 5 - DNA, 34 bp, Artificial Sequence
  Description of Artificial Sequenceprimer
cgaagcttggacttccttttttgcggaaattttc

SEQ ID NO: 6 - DNA, 29 bp, Artificial Sequence
  Description of Artificial Sequenceprimer
cgggatccctttgccctgaactgccccca

SEQ ID NO: 7 - DNA, 21 bp, Artificial Sequence
  Description of Artificial Sequenceprimer
tgcgaatgcaaatcactagaa

SEQ ID NO: 8 - DNA, 20 bp, Artificial Sequence
  Description of Artificial Sequenceprimer
gaacatgtcccaacatgttg